(19) United States
(12) Patent Application Publication
(10) Pub. No.: US 2009/0032027 A1
(43) Pub. Date: Feb. 5, 2009

(54) Multiple balloon endotracheal tube cuff

(76) Inventors: Brian Christopher McCachren, Cumming, GA (US); Scott Matthew Teixeira, Cumming, GA (US); Brian Joseph Cuevas, Cumming, GA (US); Alison Salyer Bagwell, Alpharetta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/881,894

(22) Filed: Jul. 30, 2007

(51) Int. Cl. A61M 16/04

(52) U.S. Cl. 128/207.15

(57) ABSTRACT

An airway ventilation device for insertion in a body cavity is provided. The airway ventilation device includes an endotracheal tube having multiple inflation cuffs. Adjacent inflation cuffs form indentation areas whereby bacterial secretions are removed through various liquid stabilizing means. An individual inflation cuff may optionally exhibit a twist in the opposite directions of an adjacent inflation cuff.

### BACKGROUND

Current endotracheal tubes and other airway sealing medical devices utilize an inflation cuff that presses against the sides of the trachea or other body cavity as a fluid barrier. The integrity of this cuff seal is critical, as there is a substantial amount of secretions that accumulate on top of the tubes during use that are harmful to the patient if allowed to enter the patient's lungs or pass beyond such other devices in other applications.

Often, during a medical procedure, the medical professional will be required to manipulate the endotracheal tube in a transverse or longitudinal manner or the patient may shift which may result in movement of the tube. During manipulation or movement of the endotracheal tube, gaps often form between the tracheal walls and the inflation cuffs. Additionally, gaps may form between the tracheal wall and the inflation cuffs because the inflation cuffs slowly deflate over time.

These gaps are particularly disturbing because they allow for the passage or aspiration of secretions into the lungs. These secretions often contain bacteria that may cause ventilator acquired pneumonia which according to some studies accounts for 7-8% of all deaths in hospital Intensive Care Units.

Several attempts have been made to prevent leakage of secretions around the inflation cuffs of endotracheal tubes into the lungs. For example, inflation cuffs have been used which expand elastically while being inflated. However, with these elastically expansible inflation cuffs, the filling pressure often exceeds the blood flow pressure of the walls of the cavity the inflation cuff is filling. This often leads to damage to the mucous membrane of the cavity. Additionally, these inflation cuffs are sometimes under inflated leading to leakage of bacteria beyond the inflation cuffs via folds that are present in the under inflated inflation cuffs.

As a solution, high volume, low pressure inflation cuffs have been used. These inflation cuffs, in their unfolded, unexpanded state, have a diameter greater than the diameter of the cavity in which it is inflated. This maintains the filling pressure of the balloon below the blood flow pressure of the walls of the cavity the inflation cuff fills.

Although high volume, low pressure inflation cuffs are generally effective, occasionally, secretions will still travel past cuffs into the lungs.

Thus, there remains a need for an economical offering additional protection against the passage of bacterial secretions into the lungs

### SUMMARY OF INVENTION

The present invention provides for an airway ventilation device for insertion into an elongate body cavity. The airway ventilation device includes an air tube having a distal end, a proximal end, and at least one inflation lumen having at least one inflation opening. The air tube has multiple adjacent inflation cuffs attached it. Each of the inflation cuffs may optionally have a twist in the opposite direction of the twist of an adjacent cuff to form an indentation area. Additionally, the airway ventilation device includes one or more liquid stabilizing means such as a superabsorbent or a suctioning device adapted to suction the indentation areas adjacent notch openings.

Desirably, the airway ventilation device may be constructed so that the second attachment zone of a first inflation cuff in comparison to the distal end of the same cuff, is located at a distance further from the distal end of the tube, and a first attachment zone of a second, adjacent inflation cuff in comparison to the proximal end of the adjacent inflation cuff, is located at a distance further from the proximal end of the tube when the second inflation cuff is located closer to the distal end of the tube than the first inflation cuff.

This configuration of inflation cuffs will allow an indentation area to form between the cuffs so that secretions may be trapped between the cuffs and optionally suctioned off or absorbed with a superabsorbent prior to the secretions traveling past the adjacent cuff or cuffs into the lungs.

Desirably, the inflation cuff is inflated by introducing a fluid through one or more openings that open up into the interior of the inflation cuff.

The inflation cuff may desirably contain polyurethane material and may desirably have a thickness between 5 and 30 microns.

Another aspect of the invention addresses an airway ventilation device for insertion into a body cavity. The airway ventilation device includes an air tube having a distal end, a proximal end, and multiple inflation openings. The air tube has multiple adjacent inflation cuffs attached to it which form indentation areas between cuffs. Additionally, the airway ventilation device includes one or more liquid stabilizing means such as a superabsorbent or a suction device adapted to suction the indentation areas adjacent notch openings.

Yet another aspect of the invention addresses an airway ventilation device for insertion into an elongate body cavity. The airway ventilation device includes an air tube having a distal end, a proximal end, and multiple inflation openings. The air tube has multiple adjacent inflation cuffs attached it. Each of the inflation cuffs has a twist in the opposite direction of the twist of an adjacent cuff. The space between the twisted inflation cuffs form an indentation area.

### BRIEF DESCRIPTION OF DRAWINGS

FIG. 1 is a perspective view of an airway ventilation device having multiple inflation cuffs and an indentation area.

FIG. 2 is a perspective view of an airway ventilation device having twisted inflation cuffs and an indentation area.

FIG. 3 is a perspective view of an airway ventilation device having multiple inflation cuffs which are collapsed upon each other.

FIG. 4 is a perspective view of an airway ventilation device having multiple inflation cuffs which are twisted and have broken longitudinal folds.

### DEFINITIONS

Superabsorbent—The term “superabsorbent” refers to a water-swellable, water-insoluble organic or inorganic material capable, under the most favorable conditions, of absorbing at least about 15 times its weight and, more desirably, at least about 25 times its weight of an aqueous solution containing 0.9 weight percent sodium chloride. Superabsorbent materials can be natural, synthetic, and modified natural polymers and materials. In addition, superabsorbent materials can be inorganic materials, such as silica gels, or organic compounds such as cross-linked polymers. Superabsorbent material may be biodegradable or non-biodegradable. Superabsorbent materials can include particles, fibers, flakes, films, foams, and the like. A material is “absorbent” if it absorbs at least five times its weight of the aqueous solution under these conditions.

### DETAILED DESCRIPTION

The airway ventilation device of the present invention provides for advanced endotracheal tube designs which are based upon the use of multiple subglottic inflation cuffs. The endotracheal tubes may utilize a liquid stabilizing means, such as a suctioning device or superabsorbent, for absorption and/or removal of secretions prior to passage into the lungs. Additionally, the endotracheal tubes may include twisted cuffs which break the longitudinal folds of the cuffs and prevent the passage of secretions into the lungs. Upon breakage of the longitudinal folds, the secretions are transported into an indentation area where the secretions are optionally absorbed and/or suctioned off through one of more notch openings prior to passage of the secretion past the multiple inflations cuffs into the lungs.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

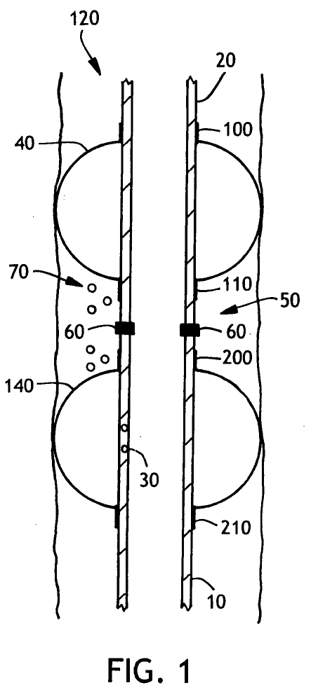

Referring to FIG. 1, an airway ventilation device for insertion into a body cavity is provided. The apparatus includes multiple inflation cuffs 40, 140 attached to an endotracheal tube 120. Although the use of more than 2 inflation cuffs is contemplated, for purposes of this discussion, a first inflation cuff 40 and second inflation cuff 140 have been illustrated. The endotracheal tube has a distal end 10, a proximal end 20, a first attachment zone 100 on the first inflation cuff 40, a second attachment zone 110 on the first inflation cuff 40, a first attachment zone 200 on the second inflation cuff 140, and a second attachment zone 210 on the second inflation cuff 140. The tube 120 may also include one or more openings 30 for introduction of fluids into the inflation cuffs 40, the openings being connected to an inflation lumen, (not shown). These fluids include gases and liquids.

The airway ventilation device, desirably an endotracheal tube, may include any flexible material which is compatible with the elongate body cavity into which it is inserted and which does not cause irritation of the cavity. These materials include, but are not limited to, latex, silicone, polyvinyl chloride, polyurethane or polytetrafluoroethylene.

The tube may be manufactured by any method known in the art of manufacturing tubing. A non-limiting example of an endotracheal manufacturing process suitable for this purpose is the coextrusion of two tubes (coextrusion being a process known and understood by those having skill in the manufacture of tubing), an outer tube and an inner tube. Additional, non-limiting examples include utilizing reinforcement inserts such as wire inside the shaft of the endotracheal tube, utilizing a spiral wound reinforcement, or utilizing a stabilizing mesh incorporated into the wall of the endotracheal tube.

Referring again to FIG. 1, the apparatus also include inflation cuffs 40, 140 attached to the endotracheal tube 120 at multiple first and second attachment zones, 100, 200 and 110, 210, respectively. The inflation cuffs may be attached to the multiple attachment zones utilizing gluing, welding, suturing or any other known method in the art for attaching an inflation cuff to an endotracheal tube. The inflation cuffs may be desirably preformed or may be alternatively formed by methods such as blow-molding, dip coating, or spin coating.

When the inflation cuffs are preformed they are preformed to exhibit dimensions greater than the diameter of the elongate body cavity in which it is to be inserted, for example, the trachea. In this regard, they are preformed to exhibit dimensions from between about 1 to about 20 percent, desirably about 1 to about 10 percent, and more desirably between about 3 percent to about 7 percent beyond the diameter of the trachea. It is also contemplated that the preformed inflation cuffs may exhibit dimension greater than 20 percent beyond the diameter of the trachea. Regardless of the diameter of the preformed inflation cuff, upon inflation, the inflation cuff should have a diameter which allows any secretions to flow into the indentation areas 50 between adjacent inflation cuffs. While these dimensions are variable, they may be readily determined by one of ordinary skill in the art utilizing conventional techniques.

Each of the preformed inflation cuffs are then each attached to the endotracheal tube shaft at least at two attachment zones, for example, a first attachment zone 100 and a second attachment zone 110.

As an alternative to preforming, the inflation cuffs may be constructed by any of the other various techniques well-known to those skilled in the art. For example without limitation, the polymer can be dip-coated on a mandrel that has a defined size and shape, desirably greater than the diameter of the tracheal cavity. When removed from the mandrel, the inflation cuffs in their uninflated or collapsed state will assume two dimensions (length and width) of the mandrel without incurring any tensional force in the polymer.

The inflation cuffs may also be formed by spin-coating in a hollow mold. When the mold is removed, as in the case of a dip-coated mandrel, the inflation cuff will assume dimensions that are the same as the interior dimensions of the hollow mold.

In addition, inflation cuffs may be formed by injection or blow molding. In this process, a pre-formed length of tubing made of the polymer is placed in a hollow mold having internal dimensions that reflect the desired dimensions of the inflation cuff to be formed. One end of the tube is sealed off and a working fluid is injected into the open end of the tube with sufficient force to cause the working fluid to expand the tubing until the wall of the tubing is in intimate contact with the inner surface of the mold. The polymer is then annealed, if desired, and cooled after which the mold is removed leaving a portion of the tubing as an inflation cuff.

The above are but a few methods of forming the inflation cuffs. Others will be apparent to those skilled in the art. All such methods are within the scope of this invention.

Various materials may be used to form the inflation cuffs. These materials include, but are not limited to, polyurethane (PU), low-density polyethylene (LDPE), polyvinyl chloride (PVC), polyamid (PA) or polyethylene teraphthalate (PETP). Additionally, copolymer admixtures for modifying the characteristics of the material may be used, for example a low density polyethylene and ethylene-vinylacetate copolymer (LDPE-EVA), or blends of the above mentioned materials (e.g. PU with PVC or PU with PA) would be considered suitable for forming the inflation cuff. Other materials would also be suitable so long as they exhibit properties enabling them to be processed into inflation cuffs having walls, desirably microthin walls on the order of about 5 to about 100 micrometers, more desirably into a range of between about 5 to about 50 micrometers, yet even more desirably between about 5 and about 20 micrometers, and yet even more desirably between about 5 and about 15 micrometers. It is also contemplated that the walls may have a thickness of less than about 5 micrometers. Additionally, although the thickness of the microthin walls may vary, it is desirable that the thickness of the microthin material remain consistent throughout the inflation cuff.

The thickness of the walls of the inflation cuff may be measured by conventional measuring techniques. These techniques include the use of LITEMATIC which is a low-contact-force (0.01 N) thickness measuring device (LITEMATIC VL-50 available from KABUSHIKI KAISHA MITSUTOYO, Japan).

It is also desirable, that upon inflation of the microthin inflation cuff, the inflation cuff will exhibit outward pressure towards the walls of the trachea. Desirably, upon inflation, pressure inside the inflation cuff will be 30 millibars or less, desirably from about 10 to about 30 millibars, and an equal amount of pressure will be exerted upon the tracheal wall. It is also contemplated that the pressure could be less than 10 millibars for short periods of time during movement of the tube or during part of the breathing cycle.

These microthin walls are desirable because, as mentioned above, the diameter of the inflation cuff is greater than the diameter of the trachea. This allows the inflation cuff, upon inflation in the trachea to inhibit and/or arrest the free flow of secretions into the lungs. A detailed discussion of this phenomenon is found in U.S. Pat. No. 6,526,977 and U.S. Pat. No. 6,802,317 both of which are incorporated herein by reference in their entireties.

Although the microthin inflation cuffs are effective in preventing the flow of secretions into the lungs, there might be situations where secretions may leak. In this regard, although unlikely, secretions may still travel past the microthin inflation cuff into the lungs. Thus, a secondary line of defense against the transmission of bacterial secretions has been developed.

In this regard, referring to FIG. 1, endotracheal tubes utilizing multiple inflation cuffs having microthin materials have been developed. Regardless of the method of formation of the cuffs, upon inflation, indentation areas 50 will be formed between adjacent inflation cuffs. Thus, if bacterial secretions flow past one inflation cuff it will flow into the indentation area rather than flow directly into the lungs.

Within each indentation area, it is contemplated that a liquid stabilizing means may also be used to prevent harmful secretions from flowing past an individual inflation cuff towards the lungs. These liquid stabilizing means include, but are not limited to, a suctioning device (not shown) and superabsorbents 70.

It is contemplated that the suctioning device may be inserted through a notch 60 on the tube and into the indentation area so that it may suction harmful secretions from the area prior to the passage of secretions into adjacent inflation cuffs and eventually into the lungs.

It is also contemplated that a superabsorbent 70 may be placed within the indentation area surrounding and/or in communication with endotracheal tube. The superabsorbent uptakes and absorbs secretions so the secretion itself does not come into contact with walls of the tracheal cavity and/or the lungs. The mechanism by which superabsorbents function is described in U.S. Pat. No. 7,179,851 which is incorporated by reference herein in its entirety.

The superabsorbent material for use with the present disclosure are preferably biodegradable and may include particles, fibers, and flakes and may be encapsulated in a form where the superabsorbant may not be directly inhaled, such as in a nonwoven, or the superabsorbent may be adhered to the body of the endotracheal tube. More desirably, the superabsorbent may include films, foams, and the like.

Conventional superabsorbent materials include crosslinked polyelectrolytes. Polyelectrolytes include both anionic and cationic polymers. Anionic polymers contain functional groups such as carboxyl, sulfonate, sulphate, sulfite, phosphate, or a mixture thereof. Examples of anionic polymers include, but are not limited to, salts or partial salts of polyacrylic acid, polyacrylamido methylpropane sulfonic acid, polyvinyl acetic acid, polyvinyl phosphonic acid, polyvinyl sulfonic acid, isobutylene-maleic anhydride copolymer, carboxymethyl cellulose, alginic acid, carrageenan, polyaspartic acid, polyglutamic acid, and copolymers or mixtures thereof. Cationic polymers contain functional groups such as primary, secondary, and tertiary amine, imine, amide, quaternary ammonium, or mixtures thereof. Examples of cationic polymers include, but are not limited to, salts or partial salts of polyvinyl amine, polydiallyl dimethyl ammonium hydroxide, polyacrylamidopropyl trimethyl ammonium hydroxide, polyamino propanol vinyl ether, polyallylamine, chitosan, polylysine, polyglutamine, and copolymers or mixtures thereof. Examples of commercially available superabsorbent materials include SXM 9543 and FAVOR 880, both available from Stockhausen Inc. in Greensboro, N.C., U.S.A., and Dow DRYTECH 2035, available from Dow Chemical Co. in Midland, Mich., U.S.A. These and other superabsorbent materials, including multicomponent superabsorbent material (i.e., superabsorbents with both anionic and cationic polymers) and biodegradable superabsorbents, are suitable for use in the invention.

It is also contemplated that after a superabsorbent absorbs secretions, that the superabsorbent may be suctioned with a suctioning device and removed from the tracheal cavity. Alternatively, the superabsorbent may be designed to have sufficient capacity to be active and capable of absorbing liquid for an amount of time corresponding to the time that the endotracheal tube is inserted inside the body cavity. For example, the superabsorent may be capable of absorbing liquid for as much as one week, several days, several hours, or several minutes. It is also contemplated that the superabsorbent may be capable of absorbing liquid for more than one week. Additionally, the superabsorbent may remove sufficient moisture from secretions to increase the viscosity of the secretions so that the secretions cannot pass by the folds of the inflation cuff and the secretions become adapted for suctioning.

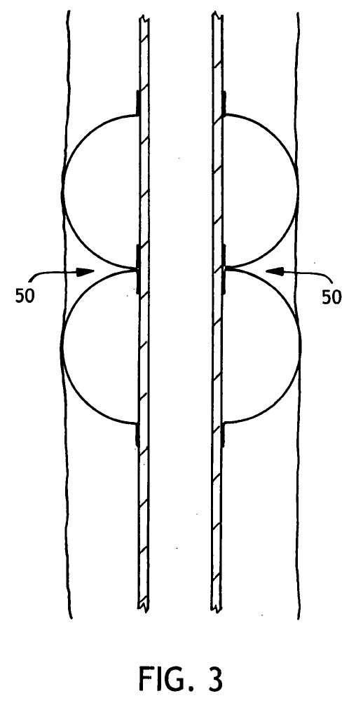

Turning now to FIG. 3, it is contemplated that adjacent inflation cuffs may be located in such close proximity to each other that they collapse upon each other when inflated. In such a situation, adjacent inflation cuffs will still form indentation areas between each other and liquid stabilizing means may still be utilized within the indentation areas for the arrest and removal of secretions prior to passage into the lungs. Thus, it is contemplated that the present invention will be effective in preventing transmission of secretions into the lungs regardless of how close or how far apart adjacent inflation cuffs are located in relation to each other.

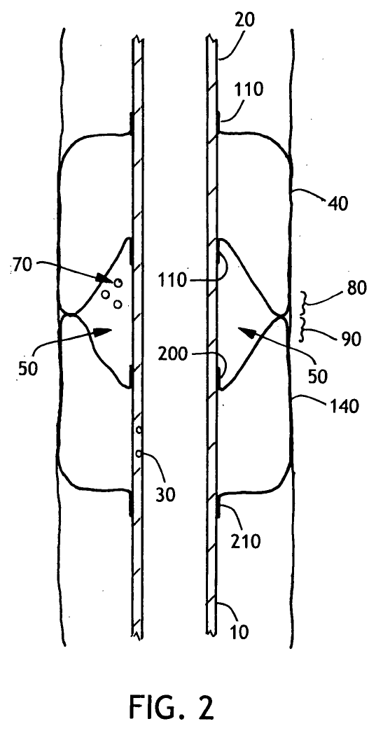

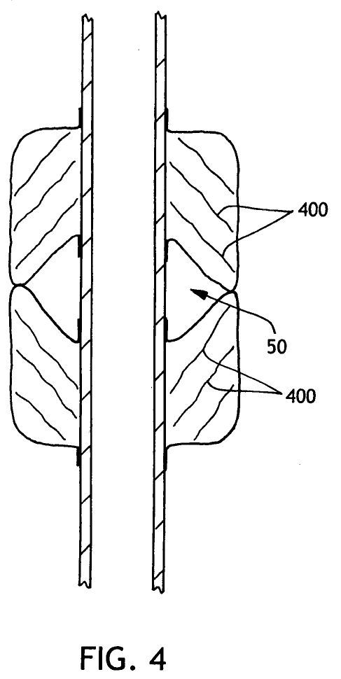

Turning now to FIG. 2, it is contemplated that adjacent inflation cuffs may be twisted when each inflation cuff is attached to the endotracheal tube. In this regard, adjacent inflation cuffs may be desirably twisted in an opposite direction of each other to form tapered substantially triangular indentation areas. The twisting of the inflation cuffs break the longitudinal folds in the inflated cuffs and transport secretions into the tapered indentation areas. An illustration of this twisting and breakage of longitudinal folds 400 is illustrated in FIG. 4. Although, the indentation areas 50 are desirably substantially triangular, they may be any shape.

Returning to FIG. 2, it is contemplated that upon “twisting” of adjacent inflation cuffs, the first cuff 40 will be located closer to the proximal end 10 of the endotracheal tube 120 than the second cuff 140 and that the second attachment zone 110 of the first inflation cuff 40, in comparison to the distal end 80 of the first inflation cuff 40 will be located a distance that is further from the distal end 10 of the endotracheal tube 120. It is also contemplated that a first attachment zone 200 of a second inflation cuff 140, in comparison to the proximal end 70 of the second inflation cuff, is located at a distance further from the proximal end 20 of the tube. Further it is contemplated that more than two inflation cuffs may be utilized and that adjacent inflation cuffs will demonstrate this same spatial arrangement.

Desirably, it is contemplated that adjacent inflation cuffs will be twisted 180 degrees or less in opposite directions, desirably 90 degrees or less in opposite directions, more desirably 60 degrees or less in opposite directions, and even more desirably 45 degrees or less in opposite directions.

It is further contemplated that secretions may remain trapped within the substantially triangular shaped indentation areas or may be removed or arrested with liquid stabilizing means such as a suctioning device and/or superabsorbents as described above.

It should be noted that while the above specific examples show particular desired embodiments of the present invention, substitution of the specific constituents of those examples with materials as disclosed herein and as are known in the art may be made without departing from the scope of the present invention. Thus, while different aspects of the present invention have been shown and described, various modifications may be made without departing from the scope of the present invention, and all such modifications and equivalents are intended to be covered.

What is claimed is:

1. An airway ventilation device for insertion into a body cavity, the device comprising:
an air tube comprising a distal end, a proximal end, and at least one inflation lumen having at least two inflation openings; and
multiple adjacent inflation cuffs attached to the air tube, each inflation cuff situated over an inflation opening, and having a twist in the opposite direction of the twist of an adjacent cuff or cuffs to form an indentation area or indentation areas;
one or more liquid stabilizing means.

2. The apparatus of claim 1 wherein the liquid stabilizing means comprises a superabsorbent.

3. The apparatus of claim 2, wherein the superabsorbent comprises a crosslinked polyelectrolyte

4. The apparatus of claim 1, wherein the liquid stabilizing means is a suctioning device, the suctioning device being adapted to suction the indentation areas.

5. The apparatus of claim 4 wherein the apparatus further comprises one or more notch openings through the air tube through which suctioning the indentation area or areas with the suctioning device is accomplished, the notch opening or openings being adjacent to the indentation area or indentation areas.

6. The apparatus of claim 1, wherein a second attachment zone of a first inflation cuff, in comparison to a distal end of the first inflation cuff, is located at a distance further from the distal end of the tube; and a first attachment zone of an second inflation cuff, in comparison to the proximal end of the second inflation cuff, is located at a distance further from the proximal end of the tube, wherein the second inflation cuff is located closer to the distal end of the tube than the first inflation cuff.

7. The apparatus of claim 1, wherein the multiple inflation cuffs are inflated through the at least two inflation openings.

8. The apparatus of claim 7, wherein the inflation of the multiple inflation cuffs is accomplished by the introduction of a fluid.

9. The apparatus of claim 1, wherein the multiple inflation cuffs comprise polyurethane material.

10. The apparatus of claim 1, wherein the thickness of the multiple inflation cuffs ranges from about 5 microns to about 30 microns.

11. An airway ventilation device for insertion into a body cavity, the device comprising:
an air tube comprising a distal end, a proximal end, and at least one inflation lumen having at least two inflation openings; and
multiple adjacent inflation cuffs attached to the air tube, each inflation cuff situated over an inflation opening, and having an indentation area or indentation areas between itself and an adjacent cuff;
one or more liquid stabilizing means.

12. The apparatus of claim 11 wherein the liquid stabilizing means comprises a superabsorbent.

13. The apparatus of claim 12, wherein the superabsorbent comprises a crosslinked polyelectrolyte.

14. The apparatus of claim 11, wherein the liquid stabilizing means is a suctioning device, the suctioning device being adapted to suction the indentation areas.

15. The apparatus of claim 14 wherein the apparatus further comprises one or more notch openings through the air tube through which suctioning the indentation area or areas with the suctioning device is accomplished, the notch opening or openings being adjacent to the indentation area or indentation areas.

16. The apparatus of claim 11, wherein the multiple inflation cuffs are inflated through the at least two inflation openings.

17. The apparatus of claim 16, wherein the inflation of the multiple inflation cuffs is accomplished by the introduction of a fluid.

18. The apparatus of claim 11, wherein the multiple inflation cuffs comprise polyurethane material.

19. The apparatus of claim 1, wherein the thickness of the multiple inflation cuffs ranges from about 5 microns to about 30 microns.

20. An airway ventilation device for insertion into a body cavity, the device comprising:
an air tube comprising a distal end, a proximal end, and at least one inflation lumen having at least two inflation openings; and
multiple adjacent inflation cuffs attached to the air tube, each inflation cuff situated over an inflation opening, and having a twist in the opposite direction of the twist of an adjacent cuff or cuffs to form an indentation area or indentation areas.
